(19) United States
(12) Patent Application Publication
(10) Pub. No.: US 2005/0143686 A1
(43) Pub. Date: Jun. 30, 2005

(54) System and method for iontophoretic transdermal delivery of one or more therapeutic agents

(75) Inventor: Daniel P. Shevlin, McKinney, TX (US)

Correspondence Address: BAKER BOTTS L.L.P., 2001 ROSS AVENUE, SUITE 600, DALLAS, TX, 75201-2980, US

(73) Assignee: EEMSO, Inc.

(21) Appl. No.: 11/059,798

(22) Filed: Feb. 17, 2005

Related U.S. Application Data
(63) Continuation of application No. 10/626,439, filed on Jul. 24, 2003.
(60) Provisional application No. 60/399,618, filed on Jul. 29, 2002.

(51) Int. Cl. A61N 1/30

(52) U.S. Cl. 604/20

(57) ABSTRACT

In one embodiment, a system for iontophoretic transdermal delivery of one or more therapeutic agents into a user's skin includes a first end including a first reservoir for containing one or more therapeutic agents, a second end including a second reservoir for containing one or more therapeutic agents, and a connecting portion coupling the first end to the second end. The connecting portion houses a self-contained power source for generating electric current, the power source having a first terminal and a second terminal. The connecting portion also houses: (1) at least a portion of a first electrode for electrically coupling the first terminal of the power source to the first reservoir, the first electrode operable to conduct electric current between the power source and the first reservoir to ionize the one or more therapeutic agents contained within the first reservoir for iontophoretic transdermal delivery into the user's skin; and (2) at least a portion of a second electrode for electrically coupling the second terminal of the power source to the second reservoir, the second electrode operable to conduct electric current between the power source and the second reservoir to ionize the one or more therapeutic agents contained within the second reservoir for iontophoretic transdermal delivery into the user's skin. The system is adapted to be used in an extended or non-extended state.

### RELATED APPLICATIONS

This application is a continuation of U.S. patent application Ser. No. 10/626,439 entitled “System and Method for Iontophoretic Transdermal Delivery of One or More Therapeutic Agents” filed Jul. 24, 2003, which claims benefit to U.S. Provisional Patent Application No. 60/399,618 entitled “System and Method for Iontophoretic Transdermal Delivery of One or More Therapeutic Agents” filed Jul. 29, 2002.

### TECHNICAL FIELD OF THE INVENTION

This invention relates generally to iontophoresis and more particularly to a system and method for iontophoretic transdermal delivery of one or more therapeutic agents.

### BACKGROUND

Iontophoresis (i.e. electrically assisted transdermal delivery of a therapeutic agent) has become an increasingly important technique for administering therapeutic agents such as analgesics, steroids, and the like. Iontophoretic transdermal delivery systems offer advantages that are not typically achievable using any other means of administration, such as introduction of the agent through mucosal absorption or skin puncture. However, such systems are known to have a number of undesirable side affects, such as skin injury ranging from redness of the skin to actual iontophoretic burns to the treated area due to fluctuations in current density. In addition, where multiple agents are contained in a reservoir of such a system, in the form of different ions, these agents may enter into competition with one another during iontophoresis, meaning that the actual transport of the charge associated with the current flow is effected through the flow of these different charge carriers. This may make it more difficult to control the dosage rates of the multiple agents individually.

### SUMMARY OF THE INVENTION

In one embodiment, a system for iontophoretic transdermal delivery of one or more therapeutic agents into a user's skin includes a first end including a first reservoir for containing one or more therapeutic agents, a second end including a second reservoir for containing one or more therapeutic agents, and a connecting portion coupling the first end to the second end. The connecting portion houses a self-contained power source for generating electric current, the power source having a first terminal and a second terminal. The connecting portion also houses: (1) at least a portion of a first electrode for electrically coupling the first terminal of the power source to the first reservoir, the first electrode operable to conduct electric current between the power source and the first reservoir to ionize the one or more therapeutic agents contained within the first reservoir for iontophoretic transdermal delivery into the user's skin; and (2) at least a portion of a second electrode for electrically coupling the second terminal of the power source to the second reservoir, the second electrode operable to conduct electric current between the power source and the second reservoir to ionize the one or more therapeutic agents contained within the second reservoir for iontophoretic transdermal delivery into the user's skin. The system is adapted to be used in an extended or non-extended state.

In another embodiment, a method for manufacturing a system for iontophoretic transdermal delivery of one or more therapeutic agents into a user's skin includes providing a first reservoir for containing one or more therapeutic agents, providing a second reservoir for containing one or more therapeutic agents, and providing a self-contained power source for generating electric current. The power source includes a first terminal and a second terminal. The method further includes providing a first electrode for electrically coupling the first terminal of the power source to the first reservoir. The first electrode conducts electric current between the power source and the first reservoir to ionize the one or more therapeutic agents contained within the first reservoir for iontophoretic transdermal delivery into the user's skin. The method further includes providing a second electrode for electrically coupling the second terminal of the power source to the second reservoir. The second electrode conducts electric current between the power source and the second reservoir to ionize the one or more therapeutic agents contained within the second reservoir for iontophoretic transdermal delivery into the user's skin. The system may be used in an extended or non-extended state.

In another embodiment, a method for delivering one or more therapeutic agents to a user through the user's skin includes positioning an iontophoretic transdermal delivery system about a portion of the user's body to receive treatment. The system may be used in an extended or non-extended state. The system includes a first end having a first reservoir for containing one or more therapeutic agents, a second end having a second reservoir for containing one or more therapeutic agents, and a connecting portion coupling the first end to the second end. The connecting portion may house a self-contained power source for generating electric current. The power source may include a first terminal and a second terminal. The connecting portion may also house a first electrode for electrically coupling the first terminal of the power source to the first reservoir. The first electrode may conduct electric current between the power source and the first reservoir to ionize one or more therapeutic agents contained within the first reservoir for iontophoretic transdermal delivery into the user's skin. The connecting portion may further include a second electrode for electrically coupling the second terminal of the power source to the second reservoir. The second electrode may conduct electric current between the power source and the second reservoir to ionize the one or more therapeutic agents contained within the second reservoir for iontophoretic transdermal delivery into the user's skin. The system may be used in an extended or non-extended state. The method further includes applying electrical current to the therapeutic agents contained in the reservoirs using the power source and delivering the therapeutic agents to the user through the user's skin in response to the electrical current.

Particular embodiments of the present invention may provide one or more technical advantages. Certain embodiments provide a simple and effective technique for administering drugs or other therapeutic agents. Certain embodiments provide two separate reservoirs each containing one or more drugs or other therapeutic agents. Certain embodiments provide the opportunity to treat more than one area of the body at a time with more than one drug or other therapeutic agent, without being limited to using ionized or ionizing agents. Certain embodiments provide an extendable, multi-function, multi-purpose system that may be used as a single bandage or as two separated bandages in delivering drugs or other therapeutic agents iontophoretically through the skin. Certain embodiments provide a fully self-contained iontophoresis system completely encased in a hypoallergenic adhesive bandage, while maintaining a shallow profile that may, in particular embodiments, be less than one sixteenth of an inch thick. Certain embodiments combine a power source, electrodes, reservoirs, and a flex-circuit to form a single applicator or, when extended, possibly to form two separate applicators. Certain embodiments provide a complete iontophoretic system in which the system and all of its components are suitable for a single patient use and are disposable after a single use.

Certain embodiments may provide all, some, or none of these technical advantages. Certain embodiments may provide one or more other technical advantages, one or more of which may be readily apparent to those skilled in the art from the figures, description, and claims included herein.

### BRIEF DESCRIPTION OF THE DRAWINGS

To provide a more complete understanding of the present invention and certain features and advantages thereof, reference is made to the following description taken in conjunction with the accompanying drawings, in which:

FIG. 1 illustrates a top view of an example system, in an extended state, for iontophoretic transdermal delivery of one or more therapeutic agents;

FIG. 2 illustrates a top view of an example system, in a non-extended state, for iontophoretic transdermal delivery of one or more therapeutic agents;

FIG. 3 illustrates an exploded perspective view, bottom side up, of an example system for iontophoretic transdermal delivery of one or more therapeutic agents;

FIG. 4A illustrates a detailed view, looking up at the top, of an example first electrode of an example power strip;

FIG. 4B illustrates a detailed view, looking down at the bottom, of an example second electrode of an example power strip;

FIG. 5A illustrates a top view of an example power strip;

FIG. 5B illustrates a cross-sectional view of the example power strip of FIG. 5A;

FIG. 6A illustrates a top view of an example system for iontophoretic transdermal delivery of one or more therapeutic agents;

FIGS. 6B and 6C illustrate cross-sectional views of the example system for iontophoretic transdermal delivery of one or more therapeutic agents of FIG. 6A;

FIG. 7 illustrates a bottom view of an example system for iontophoretic transdermal delivery of one or more therapeutic agents, depicting a pair of exposed reservoir pads and surrounding gaskets when the system is in an extended state;

FIG. 8 illustrates a bottom view of an example system for iontophoretic transdermal delivery of one or more therapeutic agents, depicting a pair of exposed reservoir pads and surrounding gaskets when the system is in a non-extended state; and

FIG. 9 illustrates an example method for treating at least one portion of a user's body with one or more therapeutic agents using an example iontophoretic transdermal delivery system.

### DESCRIPTION OF EXAMPLE EMBODIMENTS

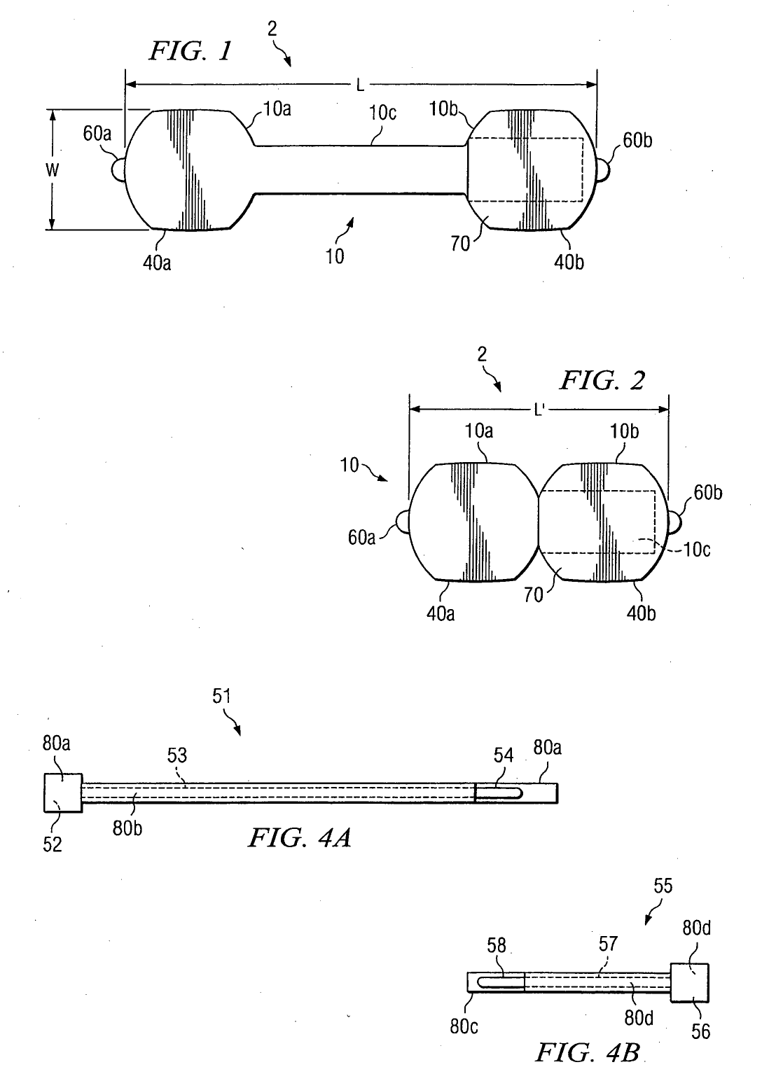

FIG. 1 illustrates a top view of an example system 2, in an extended state, for iontophoretic transdermal delivery of one or more therapeutic agents. In certain embodiments, system 2 provides an extendable, multi-function, multi-purpose iontophoretic transdermal delivery system that may be used as a single bandage or as two separated bandages in delivering one or more therapeutic agents iontophoretically through the user's skin. In a particular embodiment, system 2 provides a complete iontophoretic system in which system 2 and all of its components are suitable for a single patient use and are disposable after a single use.

As shown in FIG. 1, system 2 includes an outer strip 10 that includes a positive end 10a associated with a positive electrode, a negative end 10b associated with a negative electrode, and a connecting portion 10c coupling positive end 10a to negative end 10b. Outer strip 10 may be made from an FDA-approved hypoallergenic material, which may be either woven or non-woven, with a hypoallergenic adhesive on its bottom surface for removably coupling the system to the user's skin. In certain embodiments, outer strip 10 is preferably soft, flexible, foldable, and moldable to the surface of the user's skin.

System 2 includes one or more reservoirs 40 containing one or more therapeutic agents for application to the user's skin. In certain embodiments, system 2 provides two separate reservoirs 40, each containing one or more therapeutic agents. The use of two separate reservoirs 40 provides the opportunity to treat more than one area of the user's body at a time and may be desirable when more than one therapeutic agent is to be applied. In certain embodiments, system 2 includes protective tabs 60 which may be made from a paper material and removably coupled to a hypoallergenic adhesive on the bottoms of reservoir gaskets associated with reservoirs 40 to protect and provide protection from the therapeutic agents in reservoirs 40 prior to application of system 2 to the user's skin. As shown in FIG. 1, in an extended state, system 2 has an extended length L and a width W. In a particular embodiment, extended length L is approximately 10.375 inches and width W is approximately 2.559 inches, although system 2 may have any suitable length and width according to particular needs.

Where system 2 is extendable from a non-extended state to an extended state shown in FIG. 1, outer strip 10 may provide a “hidden” pocket 70 on one end, such as negative end 10b, to house connecting portion 10c and associated components when system 2 is in a non-extended state. In certain embodiments, such components may include a power source, electrodes, and an associated flex-circuit as discussed in more detail below.

FIG. 2 illustrates a top view of example system 2 in a non-extended state. As shown in FIG. 2, in a non-extended state, system 2 has a non-extended length L′. In a particular embodiment, non-extended length L′ is approximately 5.500 inches, although system 2 may have any suitable non-extended length according to particular needs. As discussed above, when system 2 is in a non-extended state, connecting portion 10c and associated components may be housed in pocket 70.

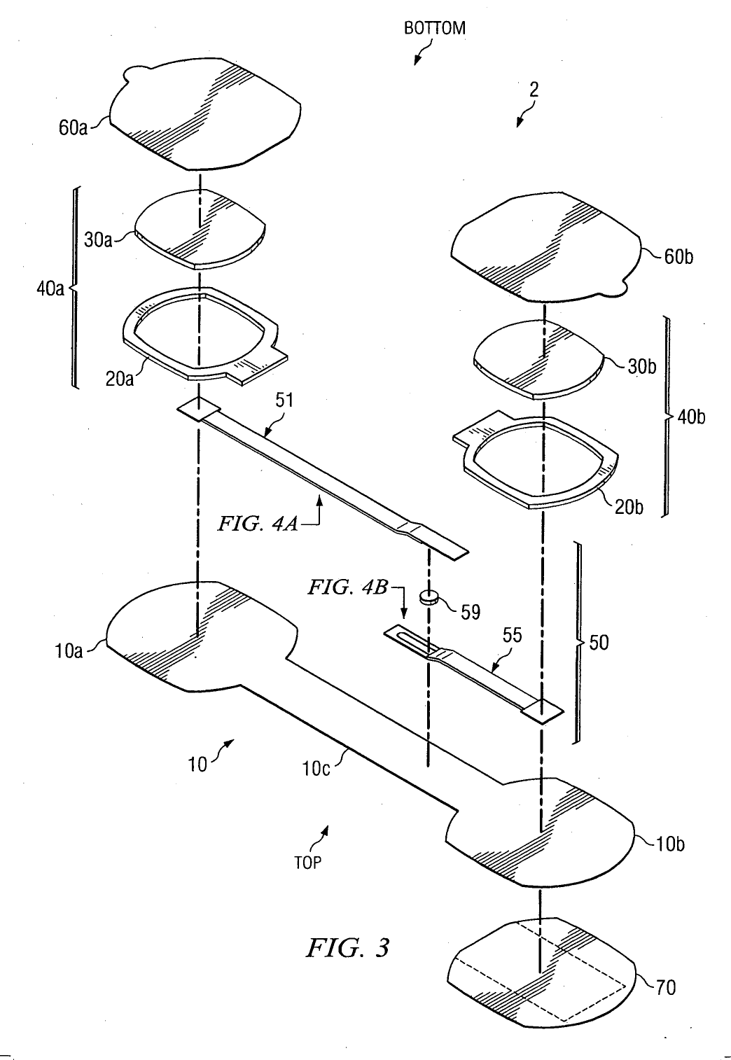

FIG. 3 illustrates an exploded perspective view, bottom side up, of example system 2. In certain embodiments, each reservoir 40 includes a reservoir gasket 20 and a reservoir pad 30. Thus, where system 2 includes two reservoirs 40a and 40b, one for each end 10a and 10b, reservoir gaskets 20a and 20b and reservoir pads 30a and 30b are provided. Reservoir gaskets 20 are used to help contain the one or more therapeutic agents within associated reservoirs 40 to prevent leakage to other parts of the user's skin during application of system 2 and subsequent treatment. In a particular embodiment, reservoir gaskets 20 may be made from a soft, flexible, foldable, FDA-approved, hypoallergenic foam material. In certain embodiments, reservoir pads 30 are used to absorb the one or more therapeutic agents to contain them in reservoirs 40 prior to treatment. In a particular embodiment, reservoir pads 30 may be made from a soft, flexible, foldable, absorbent, FDA approved, hypoallergenic material.

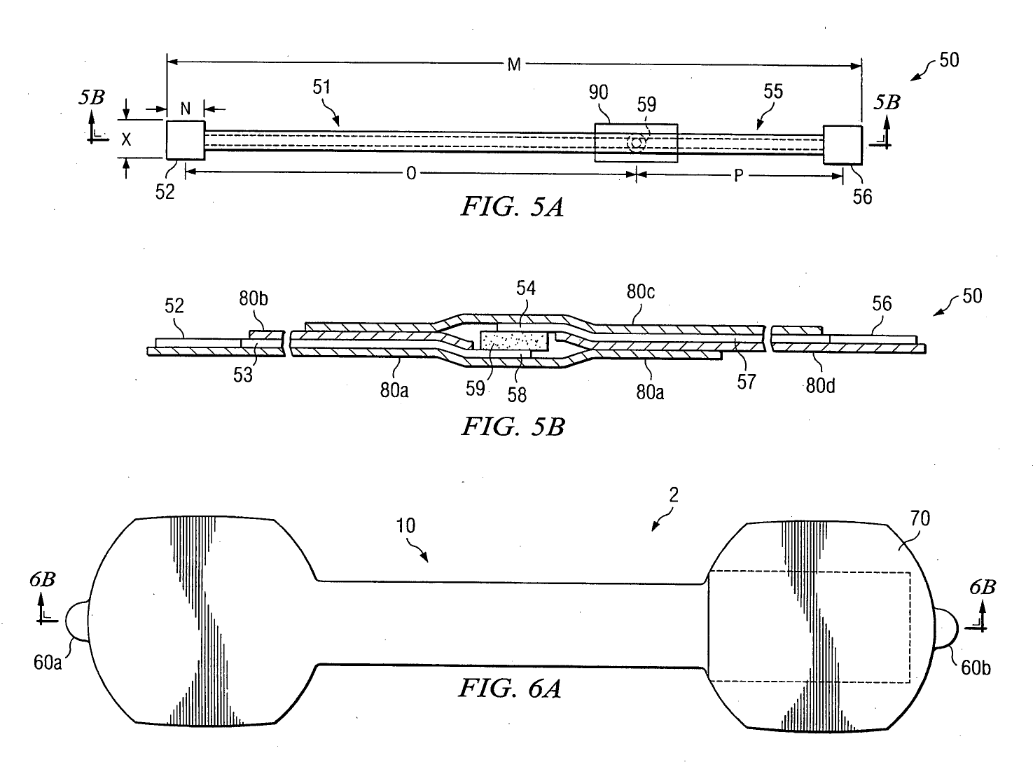

In certain embodiments, system 2 includes a power strip 50 having a first electrode 51, a second electrode 55, and a power source 59 to positively and negatively ionize or otherwise charge the one or more therapeutic agents within reservoir pads 30 for delivery of the therapeutic agents through the user's skin. Power strip 50 is described more fully below with reference to FIGS. 4A, 4B, 5A, and 5B. Power source 59 may have a negative terminal and a positive terminal. Power source 59 may be self-contained. For example, in a particular embodiment, power source 59 is a 1.55 volt battery. Although example system 2 is discussed as having a power source 59 for ionizing the therapeutic agents, the present invention contemplates using system 2 without using ionized or ionizing therapeutic agents.

Where system 2 is in a non-extended state, “hidden” pocket 70 may be used to house the flex-circuit portions of power strip 50, discussed below with reference to FIGS. 4A, 4B, and 5, and power source 59. In certain embodiments, all components associated with connecting portion 10c fold over themselves in the direction of negative end 10b and, once in their folded state, slide into hidden pocket 70. Although hidden pocket 70 is described as associated with negative end 10b, the components associated with connecting portion 10c may fold in either direction (i.e. toward positive end 10a or negative end 10b) depending on the configuration of system 2. To extend system 2 from a non-extended state, the components are removed from hidden pocket 70 and unfolded in a reverse manner. Among other benefits, the extendable nature of certain embodiments of system 2 allows for a sequential separation of the therapeutic agents to be administered through the user's skin by way of the electric current from power source 59. Another benefit of the extendable nature of certain embodiments of system 2 is the ability to treat two areas of a user's body at one time, with the same or different therapeutic agents.

FIG. 4A illustrates a detailed view, looking up at the top, of an example first electrode 51 of an example power strip 50. In certain embodiments, first electrode 51 includes a first electrode end 52 and a first conductor 53 coupled to first electrode end 52. First conductor 53 and first electrode end 52 may each comprise an electrically conductive material such as silver, copper, silver chloride, zinc, or any other material suitable to conduct and deliver an electrical current to the therapeutic agents. In certain embodiments, first conductor 53 and first conductor end 54 may comprise part of a flex circuit portion of power strip 50. First conductor 53 may be disposed between insulating layers 80a and 80b. Insulating layer 80b may be of a sufficiently shorter length than insulating layer 80a such that first conductor end 54 is at least partially uncovered to enable proper electrical contact with power source 59. The contact between power source 59 and first conductor end 54 is described more fully below with reference to FIG. 5B. Insulating layer 80a may be disposed on the top side (i.e. the side facing the viewer in FIG. 4A) of first electrode end 52. In certain embodiments, insulating layers 80 may include any appropriate soft, flexible insulating material.

FIG. 4B illustrates a detailed view, looking down at the bottom, of an example second electrode 55 of an example power strip 50. Second electrode 55 includes a second electrode end 56 and a second conductor 57 coupled to second electrode end 56. Second conductor 57 and second electrode end 56 may each comprise an electrically conductive material such as silver, copper, silver chloride, zinc, or any other material suitable to conduct and deliver an electrical current to the therapeutic agents. In certain embodiments, second conductor 57 and second conductor 58 may comprise part of the flex-circuit portion of power strip 50. Second conductor 57 may be disposed between insulating layers 80c and 80d. Insulating layer 80d may be of a sufficiently shorter length than insulating layer 80c such that second conductor end 58 is at least partially uncovered to enable proper electrical contact with power source 59. The contact between power source 59 and second conductor end 58 is described more fully below with reference to FIG. 5B. Insulating layer 80d may be disposed on the top side (i.e. the side facing away from the viewer in FIG. 4B) of second electrode end 56. In certain embodiments, insulating layers 80 may include any appropriate soft, flexible insulating material.

FIG. 5A illustrates a top view of an example power strip 50 and its associated components. In certain embodiments, power source 59 is a self-contained power source, such as a battery, that may lie within and be insulated by a protective covering 90. In a particular embodiment, protective covering 90 may be made from a polymer or gel-like substance, although any appropriate insulating material may be used.

As shown in FIG. 5A, in certain embodiments power strip 50 has an extended-state length M. In a particular embodiment, length M is approximately 8.3438 inches, although power strip 50 may have any suitable length according to particular needs. Furthermore, first electrode end 52 and second electrode end 56 of first electrode 51 and second electrode 55, respectively, each have a length N and a width X. In certain embodiments, first electrode end 52 and second electrode end 56 are substantially square in shape, such that length N and width X are substantially the same. In a particular embodiment, length N and width X of first electrode end 52 and second electrode end 56 are each approximately 0.4375 inches, although electrodes 52 and 53 may have any suitable lengths and widths according to particular needs. The distance from an approximate centerline of first electrode end 52 to an approximate centerline of power source 59 is represented by extended-state length O. The distance from an approximate centerline of second electrode end 56 to and the approximate centerline of power source 59 is represented by extended-state length P. In a particular embodiment, length O is approximately 5.3875 inches and length P is approximately 2.5188 inches, although lengths O and P may be any suitable lengths according to particular needs.

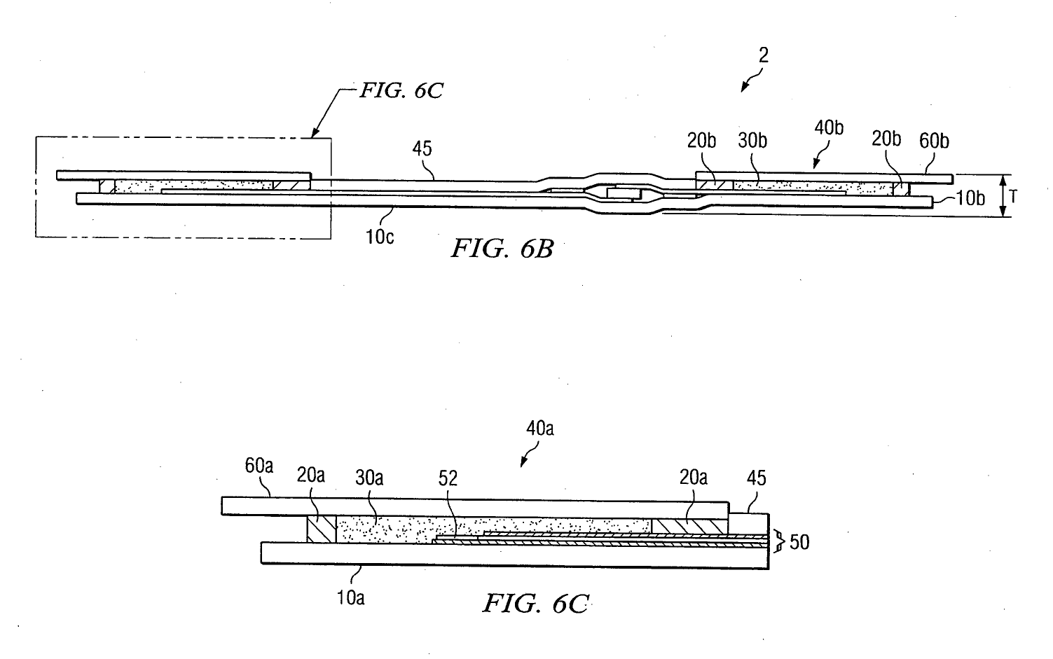

FIG. 5B illustrates a cross-sectional view of an example power strip 50 and its associated components. FIG. 5B shows the details of the various layers of certain embodiments of power strip 50, including insulating layers 80. In certain embodiments, insulating layers 80a and 80c cover first electrode end 52 and second electrode end 56 on only one side, as described above with reference to FIGS. 4A and 4B, such that first electrode end 52 and second electrode end 56 make sufficient electrical contact with reservoirs pads 30, as shown in FIG. 6C described below. FIG. 5B shows first conductor end 54 and second conductor end 58 each extending beyond layers 80b and 80d, respectively, such that first conductor end 54 and second conductor end 58 each make sufficient electrical contact with power source 59.

FIG. 6A illustrates a top view of example system 2 in an extended state. FIGS. 6B and 6C illustrate cross sectional views of example system 2 cut along section B-B of FIG. 6A.

FIG. 6B illustrates system 2 including a protective covering 45, preferably made of a hypoallergenic woven or non-woven material, to protect the self-contained power source 59 and power strip 50 from the user's skin and also to further prevent battery leakage, should it occur, into the user's skin. The positioning of reservoir gasket 20b, reservoir pad 30b, and protective tab 60b, according to a particular embodiment, is also illustrated. In certain embodiments, system 2 maintains a shallow profile. In a particular embodiment, for example, thickness T of system 2 is less than approximately one-sixteenth of an inch, although system 2 may have any suitable thickness according to particular needs.

In certain embodiments, as shown in FIG. 6C, first electrode end 52 is positioned such that it makes sufficient electrical contact with reservoir pad 30a to enable the transfer of electrical current to reservoir pad 30a. As discussed above, protective tab 60a may be made from paper removably coupled to adhesive on the bottom of reservoir gasket 20a to protect and provide protection from the therapeutic agents in reservoir pad 30a prior to application of system 2 to the users skin. When protective tab 60a is removed, reservoir pad 30a and the associated therapeutic agents may make sufficient contact with the user's skin to allow the electrical current flowing from power source 59 through first electrode end 52 to flow through reservoir pad 30a to ionize the therapeutic agents contained in reservoir 40a so as to facilitate absorption of the one or more associated therapeutic agents through the user's skin. While the details of the positive portion of system 2 are illustrated and described with reference to FIGS. 6B and 6C, the operation and arrangement of components on the opposing negative portion of system 2, such as second electrode end 56, reservoir gasket 20b, reservoir pad 30b, and protective tab 60b, are substantially similar.

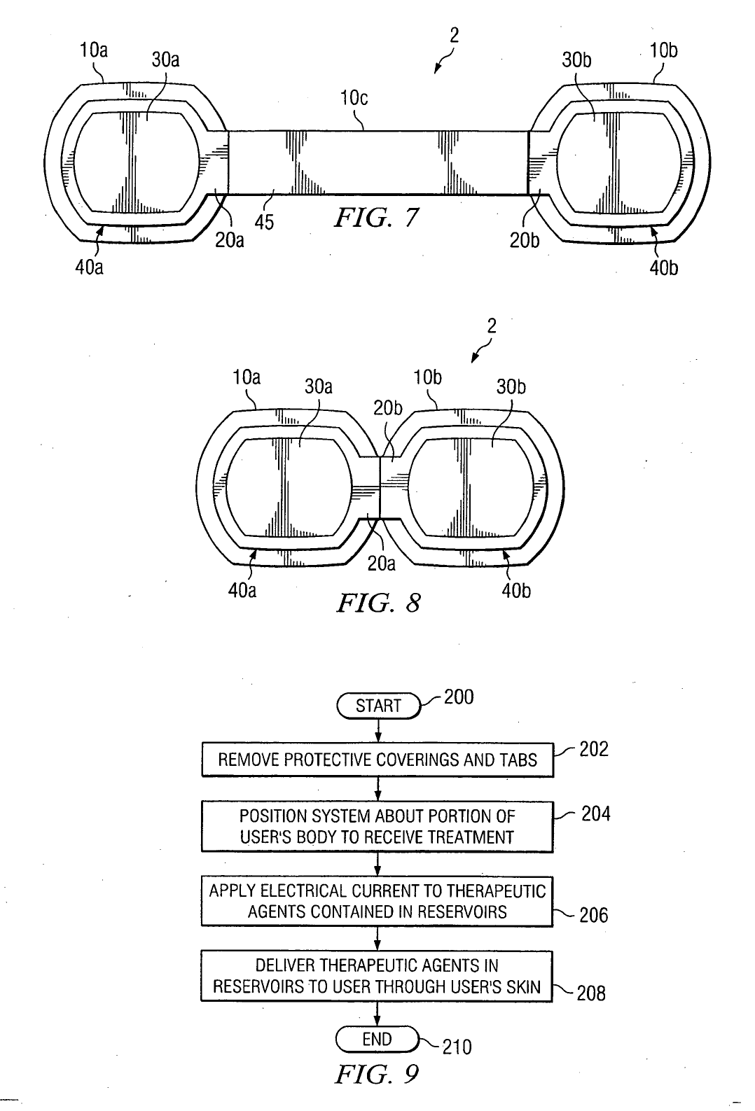

FIG. 7 illustrates a bottom view of example system 2, depicting a pair of exposed reservoir pads 30 (i.e. with protective tabs 60 removed), reservoir gaskets 20, and protective covering 45 when system 2 is in an extended state. The details of power strip 50, which is hidden behind protective covering 45, reservoir gaskets 20, and reservoir pads 30, are omitted for clarity.

FIG. 8 illustrates a bottom view of example components of example system 2, depicting a pair of exposed reservoir pads 30 and surrounding reservoir gaskets 20 when system 2 is in a non-extended state. As described above, in certain embodiments, all components associated with connecting portion 10c, including protective covering 45 and portions of power strip 50 including power source 59, fold over themselves in the direction of negative end 10b and, once in their folded state, slide into hidden pocket 70. The components associated with connecting portion 10c may fold either towards negative end 10b or positive end 10a depending on the configuration of system 2. Hidden pocket 70, and the components of power strip 50 which fold into pocket 70 when system 2 is in its non-extended state, are omitted for clarity.

FIG. 9 illustrates an example method for treating at least one portion of a user's body with one or more therapeutic agents using an example iontophoretic transdermal delivery system 2. The example method begins at step 202, where protective covering 45 and protective tabs 60 are removed. At step 204, system 2 is positioned about the portion of the user's body that is to receive the therapeutic agents. At step 206, an electrical current is applied to the therapeutic agents contained in reservoirs 40, using power source 59 for example. At step 208, the therapeutic agents are delivered to the user through the user's skin.

Although an example method is illustrated, the present invention contemplates two or more steps taking place substantially simultaneously or in a different order. In addition, the present invention contemplates using methods with additional steps, fewer steps, or different steps, so long as the steps remain appropriate for using an iontophoretic transdermal delivery system 2 for delivery of one or more therapeutic agents to at least one portion of user's body.

Furthermore, although the present invention has been described with several embodiments, a multitude of changes, substitutions, variations, alterations, and modifications may be suggested to one skilled in the art, and it is intended that the invention encompass all such changes, substitutions, variations, alterations, and modifications as fall within the spirit and scope of the appended claims.

1. A system for iontophoretic transdermal delivery of one or more therapeutic agents into a user's skin, comprising:
a first end comprising a first reservoir adapted to contain one or more therapeutic agents;
a second end comprising a second reservoir adapted to contain one or more therapeutic agents;
the first and second reservoirs each comprising:
a reservoir pad adapted to absorb the therapeutic agents to be delivered to the user; and
a foam reservoir gasket adapted to help contain the therapeutic agents contained in the reservoir pad;
a battery for generating electric current, the battery positioned between the first and second reservoirs and comprising a bottom terminal directed toward a bottom surface of the system and a top terminal directed toward a top surface of the system;
a first substantially flat, substantially straight electrode extending in a first direction from the battery, the first electrode comprising a first end that is electrically coupled to the top terminal of the battery and comprising a second end that is electrically coupled to the one or more therapeutic agents contained in the reservoir pad of the first reservoir, the first electrode operable to conduct electric current between the battery and the first reservoir to promote iontophoretic transdermal delivery of the therapeutic agents contained in the first reservoir into the user's skin; and
a second substantially flat, substantially straight electrode extending in a second direction from the battery, the second electrode comprising a first end that is electrically coupled to the bottom terminal of the battery and comprising a second end that is electrically coupled to the one or more therapeutic agents contained in the reservoir pad of the second reservoir, the second electrode operable to conduct electric current between the battery and the second reservoir to promote iontophoretic transdermal delivery of the therapeutic agents contained in the second reservoir into the user's skin;
the first and second electrodes and the battery positioned along a substantially straight line between the first end and the second end;
the first and second electrodes lying in substantially the same plane.

2. The system of claim 1, wherein:
the first electrode and the second electrode are each physically connected to the battery; and
a portion of one or both of the first or second electrodes extends from the plane in which the first and second electrodes substantially lie to physically connect to the battery.

3. The system of claim 1, wherein the first and second reservoirs are adapted to each deliver at least one therapeutic agent to the user's skin substantially simultaneously.

4. The system of claim 1, further comprising a protective tab associated with each reservoir, each tab adapted to be removably coupled to a hypoallergenic adhesive associated with its reservoir and further adapted to protect and provide protection from the therapeutic agents in its reservoir during application of the system to the user's skin, the hypoallergenic adhesive adapted to removably couple the system to a portion of the user's body.

5. The system of claim 1, wherein the first electrode, the second electrode, and the battery comprise a flex-circuit.

6. The system of claim 5, further comprising a hidden pocket disposed on the first or second end and adapted to house at least a portion of the flex-circuit when the system is in a non-extended state.

7. The system of claim 1, wherein the first electrode, the second electrode, and the battery are covered by at least one layer of insulating material to protect the user's skin.

8. A method for manufacturing a system for iontophoretic transdermal delivery of one or more therapeutic agents into a user's skin, comprising:
providing a first reservoir adapted to contain one or more therapeutic agents;
providing a second reservoir adapted to contain one or more therapeutic agents;
providing for each reservoir a reservoir pad adapted to absorb the therapeutic agents to be delivered to the user;
providing for each reservoir a foam reservoir gasket adapted to help contain the therapeutic agents contained in the reservoir pad;
providing a battery for generating electric current, the battery positioned between the first and second reservoirs and comprising a bottom terminal directed toward a bottom surface of the system and a top terminal directed toward a top surface of the system;
providing a first substantially flat, substantially straight electrode extending in a first direction from the battery, the first electrode comprising a first end that is electrically coupled to the top terminal of the battery and comprising a second end that is electrically coupled to the one or more therapeutic agents contained in the reservoir pad of the first reservoir, the first electrode operable to conduct electric current between the battery and the first reservoir to promote iontophoretic transdermal delivery of the therapeutic agents contained in the first reservoir into the user's skin;
providing a second substantially flat, substantially straight electrode extending in a second direction from the battery, the second electrode comprising a first end that is electrically coupled to the bottom terminal of the battery and comprising a second end that is electrically coupled to the one or more therapeutic agents contained in the reservoir pad of the second reservoir, the second electrode operable to conduct electric current between the battery and the second reservoir to promote iontophoretic transdermal delivery of the therapeutic agents contained in the second reservoir into the user's skin;
positioning the first and second electrodes and the battery along a substantially straight line between the first end and the second end; and
positioning the first and second electrodes lying in substantially the same plane.

9. The method of claim 8, further comprising physically connecting the first electrode and the second electrode to the battery, wherein a portion of one or both of the first or second electrodes extends from the plane in which the first and second electrodes substantially lie to physically connect to the battery.

10. The method of claim 8, wherein the first and second reservoirs are adapted to each deliver at least one therapeutic agent to the user's skin substantially simultaneously.

11. The method of claim 8, further comprising providing a protective tab associated with each reservoir, each tab adapted to be removably coupled to a hypoallergenic adhesive associated with its reservoir and further adapted to protect and provide protection from the therapeutic agents in its reservoir during application of the system to the user's skin, the hypoallergenic adhesive adapted to removably couple the system to a portion of the user's body.

12. The method of claim 8, wherein the first electrode, the second electrode, and the battery comprise a flex-circuit.

13. The method of claim 12, further comprising providing a hidden pocket disposed on the first or second end and adapted to house at least a portion of the flex-circuit when the system is in a non-extended state.

14. The method of claim 8, further comprising providing at least one layer of insulating material disposed about the first electrode, the second electrode, and the battery, the insulating material adapted to protect the user's skin.

15. A method for delivering one or more therapeutic agents to a user through the user's skin, comprising:
positioning an iontophoretic transdermal delivery system on a portion of the user's body to receive treatment, the system comprising:
a first end comprising a first reservoir adapted to contain one or more therapeutic agents;
a second end comprising a second reservoir adapted to contain one or more therapeutic agents;
the first and second reservoirs each comprising:
a reservoir pad adapted to absorb the therapeutic agents to be delivered to the user; and
a foam reservoir gasket adapted to help contain the therapeutic agents contained in the reservoir pad;
a battery for generating electric current, the battery positioned between the first and second reservoirs and comprising a bottom terminal directed toward a bottom surface of the system and a top terminal directed toward a top surface of the system;
a first substantially flat, substantially straight electrode extending in a first direction from the battery, the first electrode comprising a first end that is electrically coupled to the top terminal of the battery and comprising a second end that is electrically coupled to the one or more therapeutic agents contained in the reservoir pad of the first reservoir, the first electrode operable to conduct electric current between the battery and the first reservoir to promote iontophoretic transdermal delivery of the therapeutic agents contained in the first reservoir into the user's skin; and
a second substantially flat, substantially straight electrode extending in a second direction from the battery, the second electrode comprising a first end that is electrically coupled to the bottom terminal of the battery and comprising a second end that is electrically coupled to the one or more therapeutic agents contained in the reservoir pad of the second reservoir, the second electrode operable to conduct electric current between the battery and the second reservoir to promote iontophoretic transdermal delivery of the therapeutic agents contained in the second reservoir into the user's skin;
the first and second electrodes and the battery positioned along a substantially straight line between the first end and the second end;
the first and second electrodes lying in substantially the same plane;
applying electrical current to any therapeutic agents contained in the reservoirs using the battery; and
delivering the therapeutic agents to the user through the user's skin in response to the electrical current.

16. The method of claim 15, wherein:
the first electrode and the second electrode are each physically connected to the battery; and
a portion of one or both of the first or second electrodes extends from the plane in which the first and second electrodes substantially lie to physically connect to the battery.

17. The method of claim 15, wherein the first and second reservoirs are adapted to each deliver at least one therapeutic agent to the user's skin substantially simultaneously.

18. The method of claim 15, wherein the system further comprises a protective tab associated with each reservoir, each tab adapted to be removably coupled to a hypoallergenic adhesive associated with its reservoir and further adapted to protect and provide protection from the therapeutic agents in its reservoir prior to application of the system to the user's skin, the hypoallergenic adhesive adapted to removably couple the system to the portion of the user's body.

19. The method of claim 15, wherein the first electrode, the second electrode, and the battery comprise a flex-circuit.

20. The method of claim 19, wherein the system further comprises a hidden pocket disposed on the first or second end and adapted to house at least a portion of the flex-circuit when the system is in a non-extended state.

21. The method of claim 15, wherein the first electrode, the second electrode, and the battery are covered by at least one layer of insulating material to protect the user's skin.
